(19) United States
(12) Patent Application Publication
(10) Pub. No.: US 2008/0114045 A1
(43) Pub. Date: May 15, 2008

(54) Process for the Production of Candesartan

(75) Inventors: Yaping Wang, Gansu Province (CN); Yonggang Li, Shanghai (CN); Yulin Li, Gansu Province (CN); Guojun Zheng, Zhejiang Province (CN); Yi Li, Gansu Province (CN); Stefan Koecher, Ulm (DE)

Correspondence Address: HEDMAN & COSTIGAN P.C., 1185 AVENUE OF THE AMERICAS, NEW YORK, NY, 10036, US

(73) Assignee: Graf-Arco-Str. 3, Ulm (DE)

(21) Appl. No.: 11/793,244

(22) PCT Filed: Dec. 15, 2005
(86) PCT No.: PCT/DE05/02267

§ 371 Date: Jun. 14, 2007

(30) Foreign Application Priority Data
Dec. 16, 2004 (DE) 102004060699.4

(51) Int. Cl. A61K 31/4184; C07D 403/02

(52) U.S. Cl. 514/381; 548/253

(57) ABSTRACT

The present invention relates to novel processes for the preparation of candesartan or of a protected form of candesartan, of a candesartan salt or of a candesartan ester; compounds which can be used in processes according to the invention, processes for their preparation, their use in processes according to the invention; a novel polymorphic form of candesartan cilexetil, a process for its preparation and its use for the production of a medicament.

The present invention relates to novel processes for the preparation of candesartan or of a protected form of candesartan, of a candesartan salt or of a candesartan ester; compounds which can be used in processes according to the invention, processes for their preparation, their use in processes according to the invention; a novel polymorphic form of candesartan cilexetil, a process for its preparation and its use for the production of a medicament.

The active compound candesartan is an angiotensin II antagonist, which inhibits the angiotensin II receptor of type 1 and has been licensed for the treatment of essential hypertension. Candesartan shows good tolerability and can be administered perorally in the form of candesartan cilexetil.

The compound candesartan (chemical name 2-ethoxy-1-[[2′-(1H-tetrazol-5-yl)biphenyl-4-yl]methyl]-1H-benzimidazole-7-carboxylic acid) and its synthesis were described for the first time in EP 0 459 136. Candesartan is customarily marketed not as the free acid, but as the 1-{[(cyclohexyl-oxy)carbonyl]oxy}ethyl ester, also called candesartan cilexetil. According to EP 0 459 136, already preformed biphenyl derivatives are used as starting materials in the preparation of candesartan.

According to the teaching of EP 0 881 212, already preformed biphenyl derivatives are also used as starting materials in the synthesis of candesartan.

CN 1 510 031 A describes a C—C coupling, in which 1-{[(cyclo-hexyloxy)carbonyl]oxy}ethyl 2-ethoxy-1-(p-halophenyl)methyl-1H-benzimidazole-7-carboxylate is reacted with 5-(2-halo-phenyl)-2-(1H)-tetrazole by means of Grignard reaction to give candesartan cilexetil. A nickel catalyst, Cl₂Ni(PPh₃)₂, is used here.

H. Matsunuga, T. Euchi, K. Nishijima, T. Enomoto, K. Sasaoki and N. Nakamura, Chemical & Pharmaceutical Bulletin 1999, 47 (2), 182-186 describe two polymorphic forms I and II of candesartan cilexetil.

Form I is obtained on carrying out the synthesis described in EP 0 459 136.

WO 2004/085426 describes a 1,4-dioxane solvate of candesartan cilexetil and further polymorphic forms III and IV of candesartan cilexetil. Accordingly, form III should be obtainable by recrystallizing any desired form of candesartan cilexetil, but not amorphous candesartan cilexetil or candesartan cilexetil of polymorphic form III, from toluene.

It is accordingly the object of the invention to make available novel processes for the preparation of candesartan in the form of the free carboxylic acid, of a salt, of an ester or of a protected form of candesartan, in particular candesartan cilexetil, and to make available a novel form of candesartan cilexetil. At the same time, if possible, the use of toxic, allergenic, carcinogenic and/or teratogenic compounds should be refrained from.

The above object is achieved by a process for the preparation of candesartan, of a protected form of candesartan, of a candesartan salt or of a candesartan ester, in particular candesartan cilexetil, which comprises the following steps:

(a) preparation and reaction of a compound of the formula (I)

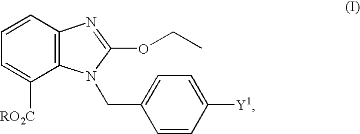

in which
R is hydrogen, an unsubstituted or substituted alkyl or aryl radical, (cyclohexyloxycarbonyloxy)ethyl, preferably methyl,
Y¹ is a group which is able to enter into a coupling reaction with formation of a C—C bond, into which a group Y² further enters,
   with a compound of the formula (II) containing the group Y²

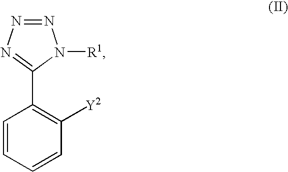

in which R¹ is a tetrazolyl protective group or hydrogen,
with formation of a protected form of candesartan or candesartan cilexetil or of another candesartan ester, and optionally
(b) conversion to candesartan, candesartan cilexetil or to a physiologically tolerable salt.

A process according to the invention is advantageous in which, in formula (I), the radical R is a C₁ to C₄-alkyl radical, in particular a methyl radical.

If, in formula (I), R is a C₁ to C₄-alkyl radical, in particular a methyl radical, then compared to a compound where R=(cyclohexyloxycarbonyloxy)ethyl, a comparatively little-functionalized compound is present, which is insensitive toward the respective reaction conditions. Because of the then comparatively low functionalization, the starting material is also less suitable for decreasing the activity of the reagents and/or catalysts used in the reaction. Such a restriction could occur, for example, by complexation of metals or metal-containing compounds employed in the reaction owing to the free electron pairs of oxygen atoms of a (cyclohexyloxycarbonyloxy)ethyl radical. In this way, the number and/or amount of by-products can be decreased and the yield increased.

If, in formula (I), R is not hydrogen, step (b) of the process according to the invention comprises the hydrolysis of the ester resulting from step (a), preferably by means of treatment with NaOH in EtOH.

The use of other means for ester hydrolysis, however, is also possible. The person skilled in the art will know to select such means.

According to the invention, step (b) can moreover comprise the reaction of candesartan with a compound of the formula (IV)

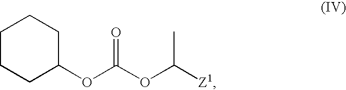

in which Z¹ is a leaving group, with formation of candesartan cilexetil, preferably in the presence of NaI and K₂CO₃.

In processes according to the invention, R¹ can be selected from hydrogen, tert-butyl and triphenylmethyl. Preferably, R¹ is triphenylmethyl.

In processes according to the invention, Y¹ can be selected from one of the following functional groups:
halogen, preferably bromine,
B(OR⁴)₂, where each of the radicals R⁴ independently of one another represents
hydrogen, alkyl, aryl or alkylaryl, preferably hydrogen,
a trialkyltin radical, or
a magnesium(II) halide radical,
   where, if Y² represents a halogen, Y¹ represents B(OR⁴), a trialkyltin or a magnesium (II) halide radical and conversely.

In processes according to the invention, Y² can represent:
halogen, preferably bromine,
B(OR⁴)₂, where each of the radicals R⁴ independently of one another represents
hydrogen, alkyl, aryl or alkylaryl, preferably hydrogen,
a trialkyltin radical, or
a magnesium(II) halide radical,
   where, if Y¹ represents a halogen, Y² represents B(OR⁴), a trialkyltin or a magnesium (II) halide radical and conversely.

In a preferred embodiment, Y¹ and Y² are selected from one of the following combinations:

### Y¹=halogen, preferably bromine, and Y²=B(OR⁴)₂, where each of the radicals R⁴ independently of one another represents hydrogen, alkyl, aryl or alkylaryl, preferably hydrogen,

### Y¹=halogen, preferably bromine, and Y²=a trialkyltin radical

### Y¹=halogen, preferably bromine, and Y²=a magnesium(II) halide radical,

### Y¹=B(OR⁴)₂, where each of the radicals R⁴ independently of one another represents hydrogen, alkyl, aryl or alkylaryl, preferably hydrogen, and Y²=halogen, preferably bromine,

### Y¹=a trialkyltin radical and Y²=halogen, preferably bromine,

### Y¹=a magnesium(II) halide radical, and Y²=halogen, preferably bromine.

If, in formula (I), R is methyl, candesartan methyl ester results, which can be converted by reaction with NaOH in EtOH to candesartan, or a candesartan salt, which in turn is convertible to candesartan cilexetil.

In a preferred embodiment, the reaction of the compound of the general formula (I) with the compound of the general formula (II) is carried out in a molar ratio of 0.2:1 to 2:1, particularly preferably of 0.3:1 to 0.8:1.

In the processes according to the invention, the reaction of the radicals Y¹ and Y² leads to a “C—C coupling”.

This C—C coupling can be carried out in the presence of Grignard reagents. These are advantageous, since they make possible a comparatively inexpensive implementation of the process according to the invention.

In processes according to the invention, one or more catalysts, preferably comprising one or more transition metals, in particular manganese, chromium, iron, cobalt, nickel or palladium, can moreover be employed. These catalysts in particular catalyze the C—C coupling reaction.

The use of catalysts of this type makes possible a particularly economical implementation of the process. The catalyst is customarily used in an amount from 0.001 mol % to 20 mol %, preferably from 0.01 to 15 and in particular 0.1 to 10 mol %, based on the molar amount of compound according to formula (I).

The catalyst(s) can be selected from MnCl₂, CrCl₃, FeCl₂, Fe(acac)₃, FeCl₃, Fe(salen)Cl, COCl₂(dppe), COCl₂(dpph), Co(acac)₂, COCl₂(dppb), Pd(PPh₃)₄, NiCl₂(PPh₃)₂.

Pd(PPh₃)₄ or NiCl₂(PPh₃)₂ is particularly preferred.

In a preferred embodiment, the catalysts used can be employed together with an activator. This activator converts the metal atoms of the catalysts to the oxidation state zero.

Examples of activators of this type are zinc (preferably in the form of zinc powder), sodium borohydride, lithium aluminum hydride or organic compounds of aluminum, magnesium or lithium (preferably butyllithium or DIBAH).

Customarily, the quantitative ratio of activator to catalyst is 25:1 to 1:1, preferably from 18:1 to 2:1.

In a further preferred embodiment, the catalysts used can be employed together with a stabilizer. This stabilizer stabilizes the metal atoms of the catalysts in the oxidation state zero.

Examples of stabilizers of this type are Lewis bases, preferably phosphanes, particularly preferably triaryl-phosphanes and trialkylphosphanes, in particular triphenylphosphane.

Customarily, the quantitative ratio of stabilizer to catalyst is 10:1 to 1:1, preferably from 5:1 to 1.5:1.

In particular, it is preferred for catalyst, activator and stabilizer to be employed together.

The use of catalysts of this type in C—C coupling reactions which contain iron, manganese, chromium or cobalt is particularly advantageous, since the metals contained therein are comparatively favorable.

In an alternative embodiment, the catalyst or the catalysts can be selected from the group consisting of the phosphane-free, preferably iron-containing catalysts. Disadvantages which accompany the use of phosphane-containing catalysts are thus avoided, namely in particular their toxicity, their tendency to combine with atmospheric oxygen, and the danger accompanying it of spontaneous combustion.

In processes according to the invention, one or more of the following solvents can moreover be employed: THF (tetrahydrofuran), THF/NMP (N-methylpyrrolidone), Et₂O (diethyl ether), DME (dimethoxyethane), benzene and toluene. THF is particularly preferred. The solvents can optionally be employed as a mixture with water.

The object is further achieved by a process for the preparation of a compound having the formula (I) defined above, which comprises the following steps:

preparation and reaction of a compound of the formula (III)

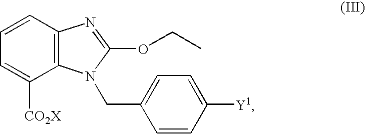

in which
Y¹ has the same meaning as above;
X is a group which is able to enter into a reaction, into which a group Z¹ further enters, with formation of an O—C bond,
with a compound of the formula (IV)

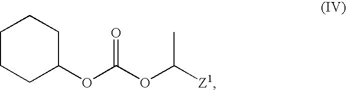

in which Z¹ is a leaving group,
with formation of a compound of the formula (I).

According to one embodiment of the invention, X can be an alkali metal or preferably hydrogen and/or Z¹ can represent a halogen, preferably iodine.

The object is further achieved by a process for the preparation of a compound having the formula (III) defined above, which comprises the following step:

preparation and deprotection of a compound of the formula (V)

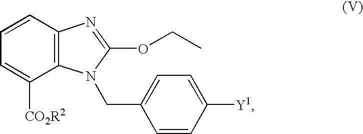

in which
Y¹ has the same meaning as above, and
R² is a group replaceable by X with formation of a compound of the formula (III), where X has the same meaning as stated above in connection with formula (III).

According to the invention, R² can be selected from one of the following functional groups: substituted or unsubstituted C₁-C₆-lower alkyl, benzyl or aryl, preferably ethyl (CH₂CH₃) and even more preferably methyl (CH₃).

The object is further achieved by a process for the preparation of a compound having the formula (V) defined above, which comprises the following step:

preparation and reaction of a compound of the formula (VI)

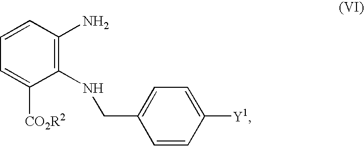

in which Y¹ and R² have the same meaning as above,
with a carbonylating reagent or preferably C(OEt)₄, with formation of a compound of the formula (V).

In such a process, Ac₂O can further be used in the reaction.

The object is further achieved by a process for the preparation of a compound of the formula (VI) as defined above, which comprises the following step:

preparation of a compound of the formula (VII)

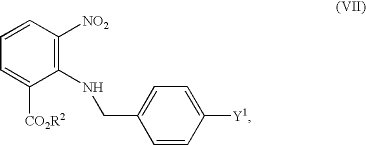

in which Y¹ and R² have the same meaning as above, and
conversion of the nitro group present therein to an amine group.

According to the invention, the conversion of the nitro group to the amine group can be brought about with the aid of base metals, catalytic hydrogenation, by electrolytic routes or preferably with the aid of SnCl₂.

The object is further achieved by a process for the preparation of a compound of the formula (VII) as defined above, which contains the following step:

preparation and deprotection of a compound of the formula (VIII)

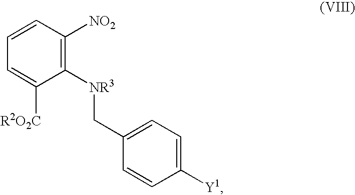

in which Y¹ and R² have the same meaning as above and in which R³ is a protective group replaceable by H,
with formation of a compound of the formula (VII) as defined above.

According to the invention, R³ can be a carboxyalkyl group, preferably a carboxy-tert-butyl group (—COOC—(CH₃)₃).

The object is further achieved by a process for the preparation of a compound of the formula (VIII), as defined above, which contains the following step:

preparation and reaction of a compound of the formula (IX)

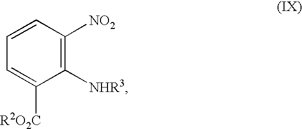

in which R² and R³ have the same meaning as above,
with a compound of the formula (X)

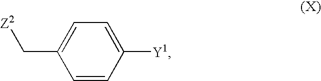

in which
Y¹ has the same meaning as above, and
Z² is a leaving group,
   with formation of a compound of the formula (VIII).

According to the invention, Z² can be selected from one of the following functional groups: Cl, I and preferably Br.

In a preferred embodiment, the reaction of a compound of the formula (IX) with a compound of the formula (X) can be carried out in the presence of basic compounds, preferably alkali metal or alkaline earth metal carbonates, in particular Na₂CO₃ or K₂CO₃.

According to the invention, the compound of the formula (VIII), (VII), (V) or (III) in each case to be prepared by the respective process according to the invention for the preparation of compounds of the formula (VII), (VI), (III) or (I) can be prepared by means of one or more of the processes according to the invention.

According to the invention, the compound of the formula (I) to be prepared in the process according to the invention for the preparation of candesartan, of a candesartan salt, of a candesartan ester or of a protected form of candesartan can further be prepared by means of one or more processes according to the invention.

The object is further achieved by an intermediate having the formula

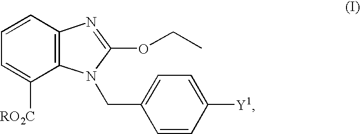

in which R is hydrogen, an unsubstituted or substituted alkyl or aryl radical, and preferably (cyclohexyloxycarbonyloxy)-ethyl, and in which Y¹ has the same meaning as above.

In a preferred intermediate of the formula (I), Y¹ is
halogen,
B(OR⁴)₂, where each of the radicals R⁴ independently of one another represent hydrogen, alkyl, aryl or alkylaryl, preferably hydrogen,
a trialkyltin radical, or
a magnesium(II) halide radical.

In a particularly preferred intermediate of the formula (I), Y¹ is Br.

Furthermore, for the intermediate of the formula (I) according to the invention the proviso preferably applies that if Y¹ is Cl, Br or I, then R is not hydrogen, ethyl or {[(cyclohexyloxy)carbonyl]oxy}ethyl. This proviso, however, does not relate to the process according to the invention.

The object is further achieved by an intermediate having the formula

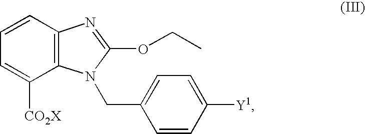

in which Y¹ and X have the same meaning as above.

Furthermore, the proviso preferably applies for the intermediate of the formula (III) according to the invention that if Y¹ is Cl, Br or I, then R is not hydrogen. This proviso, however, does not relate to the process according to the invention.

The object is further achieved by an intermediate having the formula

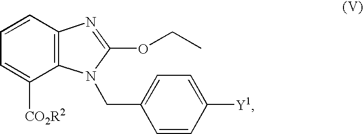

in which Y¹ and R² have the same meaning as above.

In a preferred intermediate of the formula (V), Y¹ is equal to Br and R² to a methyl group or C₃-C₆-lower alkyl group.

Furthermore, the proviso preferably applies for the intermediate of the formula (V) according to the invention that if Y¹ is Cl, Br or I, then R² is not ethyl. This proviso, however, does not relate to the process according to the invention.

The object is further achieved by an intermediate having the formula

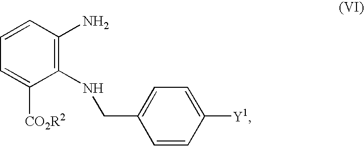

in which Y¹ and R² have the same meaning as above.

In a preferred intermediate of the formula (VI), Y¹ is equal to Br and R² to a methyl group or C₃-C₆-lower alkyl group.

Furthermore, the proviso preferably applies for the intermediate of the formula (VI) according to the invention that if Y¹ is Cl, Br or I, then R² is not ethyl. This proviso, however, does not relate to the process according to the invention.

The object is further achieved by an intermediate having the formula

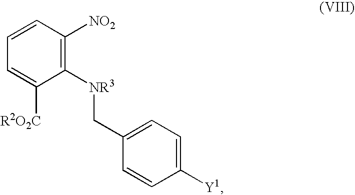

in which Y¹, R² and R³ have the same meaning as above.

In a preferred intermediate of the formula (VIII), Y¹ is equal to Br, R² to a methyl group or C₃-C₆-lower alkyl group and R³ to a carboxyalkyl group, preferably a carboxy-tert-butyl group (COOC(CH₃)₃).

Furthermore, the proviso preferably applies for the intermediate of the formula (VIII) according to the invention that if Y¹ is Cl, Br or I, then R² is not ethyl and R³ is not carboxy-tert-butyl. This proviso, however, does not relate to the process according to the invention.

According to the invention, the object is moreover achieved by use of the intermediates according to the invention and/or of the compound (VII) in processes for the preparation of candesartan, candesartan esters or candesartan cilexetil, where preferably R²=methyl and Y¹=Br.

The object is moreover achieved by a process for the preparation of a polymorphic form of candesartan cilexetil, comprising:
treatment of candesartan cilexetil with dichloromethane and diethyl ether with obtainment of a clear solution,
concentration of the clear solution and precipitation of candesartan cilexetil.

The object is further achieved by the polymorphic form of candesartan cilexetil which is obtainable by means of the process according to the invention.

The object is achieved by means of a polymorphic form of candesartan cilexetil which can be described by means of one or more of the following physical parameters:
signals in the X-ray powder diffractogram using Cu—Kα radiation expressed in 2θ at 7.32, 8.20, 9.10, 14.68, 18.88, 24.18°, where all values preferably include a standard deviation of ±0.2°;
spacings of the lattice planes d determined by means of XRD=12.065, 10.773, 9.711, 6.029, 4.696, 3.678 Å (Angstroms), where all values preferably include a standard deviation of ±0.1 Å;
a melting point determined by means of DSC at approximately 130.7° C., and/or
a characteristic absorption band in the IR spectrum at approximately 1733 cm⁻¹.

In the polymorphic form of candesartan cilexetil according to the invention, in each case relative signal intensities of the signals of approximately 100, 29.6, 20.2, 45.2, 20.5, 11.4 can be observed in the X-ray powder diffractogram.

The object is finally achieved by preparation of the polymorphic form of candesartan cilexetil according to the invention by means of the process according to the invention.

According to the invention, the polymorphic form of candesartan cilexetil according to the invention can be used for the production of a medicament.

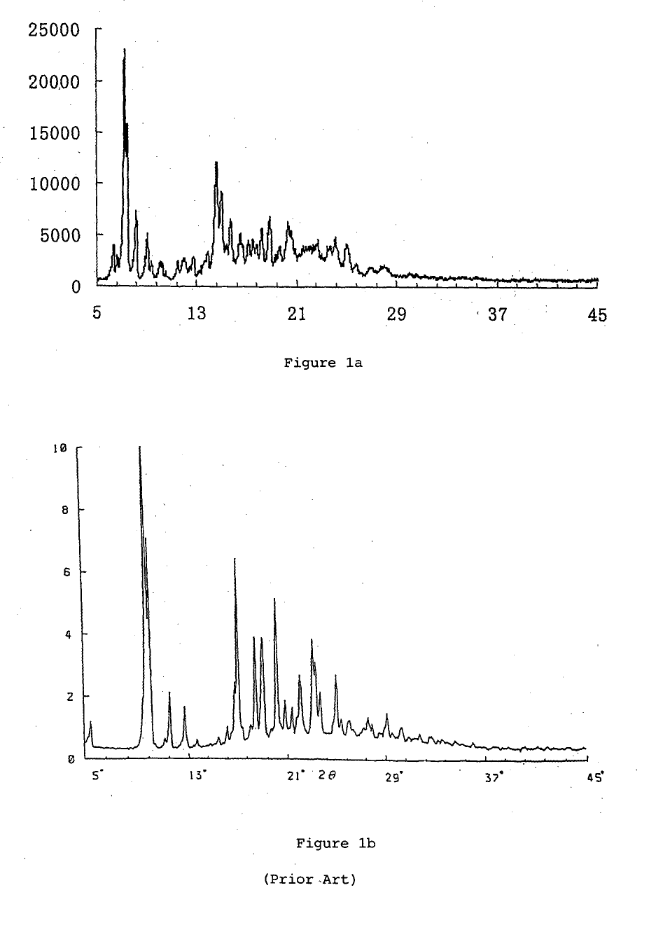

The invention is illustrated in more detail below with the aid of FIGS. 1 to 4 and with the aid of working examples.

The FIGS. 1 to 4 show, in

FIG. 1a an X-ray powder diffractogram of the polymorphic form of candesartan cilexetil according to the invention,

FIG. 1b an X-ray powder diffractogram of candesartan cilexetil of the polymorphic form I according to the prior art,

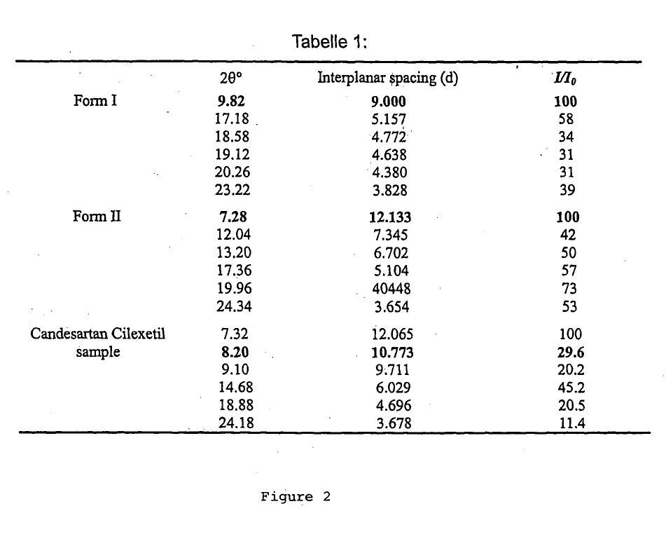

FIG. 2 a table from which the 2θ values and lattice spacings d of the polymorphic candesartan cilexetil forms I and II according to the prior art and of the polymorphic candesartan cilexetil form according to the invention are evident,

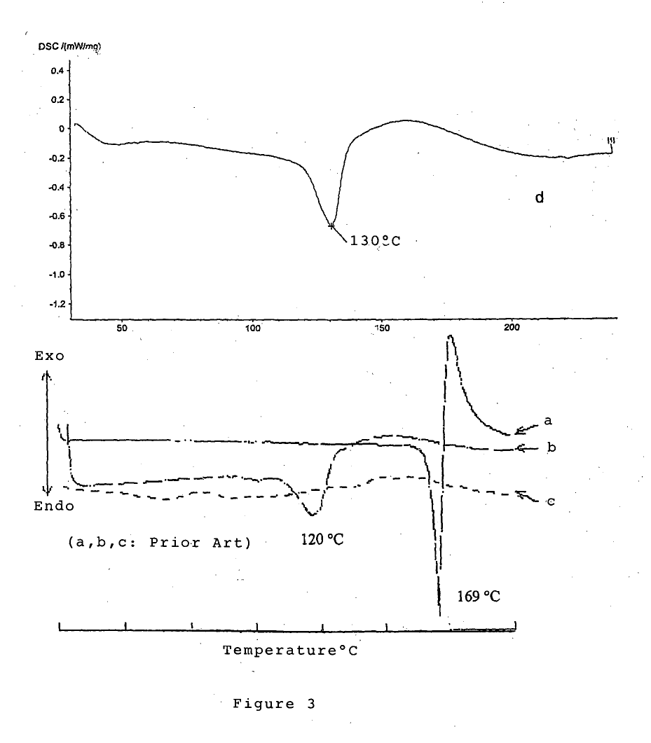

FIG. 3 a DSC curve “d” of the polymorphic form according to the invention and DSC curves of the polymorphic forms I and II (“a” and “b”) and of amorphous candesartan cilexetil (“c”) according to the prior art, and

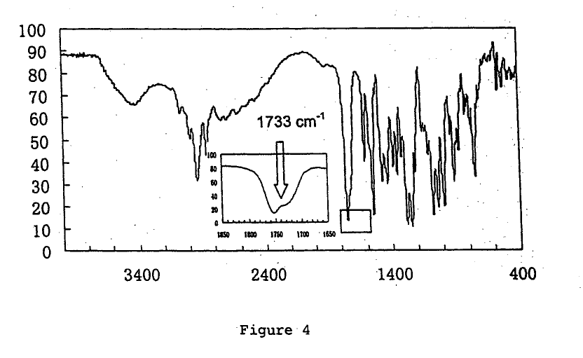

FIG. 4 an IR spectrum of the polymorphic form according to the invention.

### WORKING EXAMPLES

The following working examples relate to the preparation of compounds (e), (g), (h), (i) and (j). The respective compounds of the formulae (e), (g), (h), (i) and (j) in each case correspond to intermediates according to the invention having the formulae (VIII), (VI), (V), (I) and (III) where Y¹=Br, R²=methyl, R³=CO₂t-Bu, X=H, R={[(cyclohexyl-oxy)carbonyl]oxy}ethyl.

The target compound candesartan or candesartan cilexetil can be prepared starting from the respective intermediate compounds, as is easy to recognize for the person skilled in the art with the aid of the working examples.

### General Reaction Conditions

All dry solvents (CH₂Cl₂, THF, Et₂O, benzene, toluene, DMF, MeCN) were dried according to standard methods, i.e. by removal of water and oxygen and distillation before use. The reactions described below were carried out, if necessary, under an inert gas atmosphere (N₂ or Ar) and monitored by means of thin layer chromatography (TLC). Diethyl ether, ethyl acetate or chloroform can be used in the extractions. The extracts were dried in a customary manner, for example with the aid of anhydrous MgSO₄ or Na₂SO₄, if not stated otherwise. The reaction products were purified, if necessary, by means of column chromatography using, for example, petroleum ether (60-90° C.)/ethyl acetate or petroleum ether (30-60° C.)/ethyl acetate as the eluents. If plates of the type GF₂₅₄ were used for the TLC, iodine or an ethanolic solution of phosphomolybdic acid was used as the means of detection. The silica gel for the chromatography (200-300 particle size) and TLC (GF₂₅₄) was produced by Qingdao Sea Chemical Factory and Yantai Chemical Factory. All solvents and reagents were of analytical or chemical purity.

The melting point determination was carried out by means of an XT₄-100× micro-melting point tester. The recording of infrared spectra was carried out with the aid of KBr pressings or PE films on Nicolet AVATAR 360 FT-IR and Nicolet NEXUS 670 FT-IR spectrometers. NMR measurements were carried out on NMR spectrometers from Varian (Mercury-300) and Bruker (AM-400) using SiMe₄ as an internal standard in CDCl₃, if not noted otherwise. LRMS were determined using an HP-5988 mass spectrometer using EI at 70 eV, if not stated otherwise. HRMS were measured using a Bruker Daltonics APEX II 47e FT-ICR mass spectrometer.

### I. Preparation of

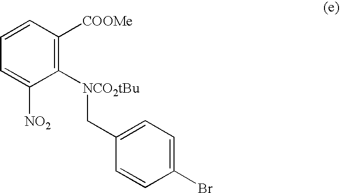

a) 3-Nitrophthalic acid (35 g) was dissolved in 215 ml of methanol, containing 20 ml of concentrated H₂SO₄, in a 500 ml round-bottomed flask. After refluxing for 24 h, the reaction mixture was concentrated in vacuo. The pH of the residue was adjusted to pH 11-12 by means of an aqueous, saturated K₂CO₃ solution. Subsequently, the reaction mixture was extracted with ethyl acetate and the pH of the aqueous phase was adjusted to pH 2-3 using concentrated hydrochloric acid. The residue was extracted (2×1000 ml of CH₂Cl₂). The organic phases were combined and washed with water and an aqueous NaCl solution, dried over MgSO₄ and concentrated. The yellow solid obtained can be directly reused without further purification; the product has the formula

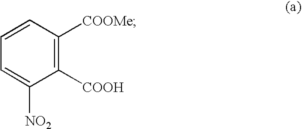

b) 30 g of compound (a) from step a) and 16 ml of SOCl₂ were dissolved in 200 ml of dry benzene in a 500 ml round-bottomed flask. The mixture was refluxed for 3 h and subsequently concentrated, a white powder being formed; the product has the formula

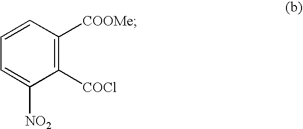

c) 30 g of compound (b) from step b) were dissolved in 120 ml of acetone in a 500 ml round-bottomed flask. An aqueous solution of 120 ml of NaN₃ (184 mmol, 12 g) was slowly added dropwise to the reaction mixture. Subsequently, the reaction mixture was stirred for one hour. The mixture was filtered, washed with ice water and dried in vacuo. The product obtained has the formula

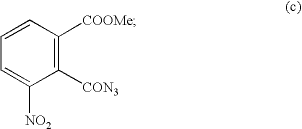

d) 28 g of compound (c) from step c) and 112 ml of tert-butanol were slowly heated in a 250 ml round-bottomed flask and refluxed for 2 h. The reaction mixture was concentrated in vacuo and purified by means of column chromatography (PE:AcOEt, 16:1 to 4:1). The crude product thus obtained can be recrystallized in methanol to give yellow crystals and has the formula

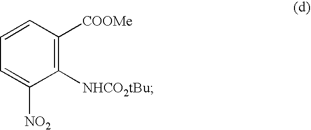

e) 10.8 g of compound (d) from step d) (36 mmol) and 4-bromo-benzyl bromide (40 mmol, 10.0 g) were dissolved in CH₃CN (200 ml) in a 500 ml round-bottomed flask. K₂CO₃ powder (36 mmol, 5.0 g) was added. The reaction mixture was refluxed for 10 h, concentrated in vacuo and extracted with ethyl acetate (2×500 ml). The extracts were washed with water and an aqueous solution of NaCl, dried over anhydrous Na₂SO₄, concentrated in vacuo and recrystallized from AcOEt/PE, which afforded the target compound (e) (15.4 g, melting point 107-108° C.) as colorless crystals. The yield was 92%. Analytical data of the target compound (e): ¹H NMR (CDCl₃, 300 MHz): δ 1.30 (9H, s, t-Bu), 3.67 (3H, s, OMe), 4.57 (2H, dd, J=14.7 Hz, CH₂), 7.01 (2H, d, J=8.1 Hz, ArH), 7.32 (2H, d, J=8.1 Hz, ArH), 7.46 (1H, t, J=8.1 Hz, ArH), 7.89 (1H, d, J=8.1 Hz, ArH), 8.00 (1H, d, J=8.1 Hz, ArH), ¹³C NMR (CDCl₃, 75 MHz): δ 27.8, 52.7, 53.1, 81.2, 121.9, 127.8, 128.1, 128.3, 131.0, 131.3, 131.6, 132.2, 134.8, 134.9, 135.3, 148.6, 153.5, 164.7; MS (EI) m/z (%): 464 (M⁺, 0.1), 365 (9), 348 (10), 316 (3), 302 (3), 235 (4), 185 (27), 169 (31), 57 (100). IR (film, cm⁻¹) λₘₐₓ=3087, 2978, 2952, 1711, 1601, 1536, 1484, 1453, 1384, 1367, 1293, 1164, 1128, 1014, 984, 864, 766, 704.

### 2. Preparation of

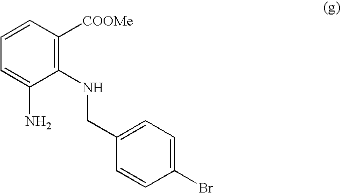

a) Compound (e) from Example 1 (3.2 mmol, 15.4 g) was dissolved in a mixture of CF₃COOH (32 ml) and CH₂Cl₂ (20 ml) in a 100 ml round-bottomed flask and stirred at room temperature for one hour. The reaction mixture was concentrated in vacuo. Subsequently, methanol (30 ml) was added and the pH was adjusted to approximately pH 10 using a concentrated aqueous NaHCO₃ solution. The yellow precipitate was filtered off and recrystallized from ethanol, which afforded the compound of the formula

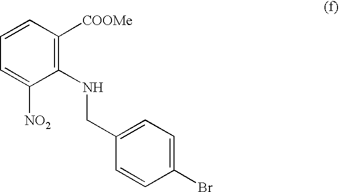

(9.45 g, melting point 111-112° C.) in the form of yellow, needle-shaped crystals. The yield was 84%. Analytical data of (f): ¹H NMR (CDCl₃, 300 MHz) δ 3.87 (3H, s, OMe), 4.09 (2H, d, J=5.1 Hz, CH₂), 6.71 (1H, t, J=7.5 Hz, ArH), 7.16 (2H, d, J=8.4 Hz, ArH), 7.45 (2H, d, J=8.4 Hz, ArH), 7.96 (1H, d, J=7.5 Hz, ArH), 8.10 (1H, d, J=7.5 Hz, ArH), 8.77 (1H, br, ArH); ¹³C NMR (CDCl₃, 75 MHz) δ 50.2, 52.4, 115.0, 116.6, 121.8, 129.6, 131.6, 131.9, 136.6, 136.9, 137.4, 145.1, 167.7; MS (EI) m/z (%): 364 (M⁺, 2), 346 (16), 302 (15), 235 (13), 207 (8), 183 (100), 169 (80), 89 (64). IR (film, cm⁻¹) λₘₐₓ=3306, 3094, 3011, 2953, 1937, 1715, 1692, 1601, 1575, 1527, 1486, 1441, 1402, 1339, 1260, 1198, 1116, 1073, 1010, 971, 893, 833, 808, 766, 734, 717, 670, 644, 589;

b) Compound (f) from step a), (7.4 g, 20 mmol) and dry ethanol (40 ml) were initially introduced in a 100 ml round-bottomed flask. SnCl₂.2H₂O (102 mmol, 23.0 g) was added in portions. The reaction mixture was heated to 80° C. for 2 h and the ethanol was removed. The residue was dissolved in ethyl acetate (60 ml) and the pH was adjusted to pH 11-12 using 4 N NaOH. The organic phase was separated off and the aqueous phase was extracted with ethyl acetate. The combined organic phases were washed with water and an aqueous NaCl solution, dried over anhydrous Na₂SO₄ and concentrated. The product obtained,

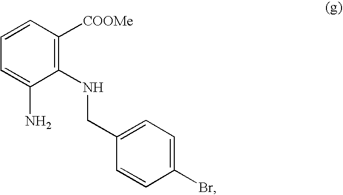

can be directly reused without further purification. Analytical data of (g): ¹H NMR (DMSO, 300 MHz) δ 3.72 (3H, s), 4.36 (2H, s), 7.08 (1H, m), 7.31 (2H, brd, J=8.1 Hz), 7.44-7.51 (4H, m), 8.69 (3H, brs); ¹³C NMR (DMSO, 75 Hz) δ 51.1, 53.2, 121.9, 123.0, 124.4, 126.4, 127.2, 131.8, 131.9, 132.0, 132.1, 132.2, 136.2, 167.8; MS (ESI) [M+H]⁺ 335.0088 (calculated 335.0089).

### 3. Preparation of

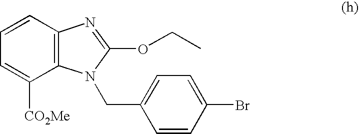

Compound (g) (31 mmol) from Example 2, C(OEt)₄ (46.5 mmol, 9.8 ml) and acetic acid (31 mmol, 1.8 ml) were mixed in a 50 ml round-bottomed flask and stirred as 80° C. for 6 h. Subsequently, the reaction mixture was concentrated, the pH was adjusted to pH 10 using a saturated NaHCO₃ solution, and the mixture was extracted with ethyl acetate (2×500 ml), dried over anhydrous Na₂SO₄ and concentrated under reduced pressure. The solid obtained was recrystallized from ethyl acetate, which afforded the target compound (h) (7.9 g, melting point 122-123° C.). Calculated starting from the amount of starting compound (f) employed (see above, Example 2), the yield was 65%. Analytical data of (h): ¹H NMR (CDCl₃, 300 MHz) δ 1.45 (3H, t, J=7.2 Hz, Me), 3.74 (3H, s, OMe), 4.63 (2H, q, J=7.2 Hz, CH₂), 5.56 (2H, s, CH₂), 6.85 (2H, d, J=9 Hz), 7.16 (1H, t, J=8.4 Hz), 7.34 (2H, d, J=9 Hz), 7.57 (1H, d, J=8.4 Hz), 7.72 (1H, d, J=8.4 Hz); ¹³C NMR (CDCl₃, 75 MHz): δ 14.6, 46.7, 52.2, 66.7, 115.5, 120.9, 122.1, 123.7, 128.2, 131.5, 136.4, 141.9, 158.6, 166.7; MS (EI) m/z (%): 388 (M⁺, 14), 361 (2), 327 (3), 299 (2), 249 (5), 221 (5), 192 (3), 169 (100), 89 (28). IR (film, cm⁻¹) λₘₐₓ=3407, 3058, 2991, 2952, 2852, 1903, 1709, 1615, 1549, 1479, 1430, 1382, 1248, 1128, 1036, 927, 869, 800, 743, 687.

### 4. Preparation of

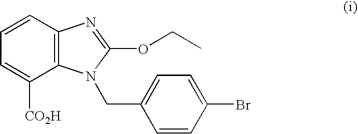

Compound (h) (10.3 mmol, 4.0 g), 1M NaOH (30 ml) and ethanol (30 ml) were mixed in a 100 ml round-bottomed flask and stirred at 80° C. for 1 h. Subsequently, the reaction mixture was concentrated under reduced pressure for the removal of the solvent. Afterward, water (50 ml) and ethyl acetate (50 ml) were added to the residue and the aqueous phase was separated off. The pH was adjusted to pH 2-3 using concentrated hydrochloric acid, which afforded compound (i) as a white solid, which was dried in vacuo; yield: 3.6 g, 95%. The compound (i) thus obtained can be directly reused without further purification. Analytical data of (i): ¹H NMR (d-DMSO, 300 MHz) δ 1.36 (3H, t, J=6.9 Hz, CH₃), 4.56 (2H, q, J=6.9 Hz, CH₂), 5.55 (2H, s, CH₂), 6.89 (2H, d, J=8.1 Hz), 7.15 (1H, t, J=7.8 Hz), 7.43-7.51 (3H, m), 7.64 (1H, d, J=8.1 Hz); ¹³C NMR (d-DMSO, 75 MHz): δ 15.0, 46.8, 67.2, 117.3, 120.9, 121.5, 122.2, 124.1, 129.2, 131.8, 132.1, 132.2, 137.7, 142.3, 158.9, 168.1; MS (ESI) [M+H]⁺ 375.0193 (calculated 375.0339).

### 5. Preparation of

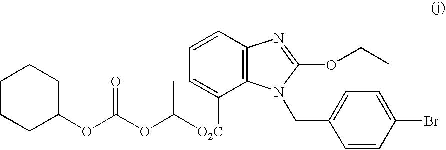

Compound (i) from Example 4 (9.7 mmol, 3.64 g), 1-{[(cyclo-hexyloxy)carbonyl]oxy}-1-iodoethane (k) from the following Example 6 (19.5 mmol, 4.05 g), anhydrous K₂CO₃ (9.7 mmol, 1.34 g), NaI (42.7 mmol, 6.4 g) and dry DMF (40 ml), were mixed in a 100 ml round-bottomed flask and stirred at 60° C. for 13 h. After it had been concentrated under reduced pressure, the reaction mixture was extracted with ethyl acetate (2×200 ml). The organic phases were separated off and washed with water and an aqueous NaCl solution, dried over anhydrous Na₂SO₄ concentrated and purified by means of column chromatography (PE:AcOEt, 16:1 to 4:1), which afforded the target compound (j) (2.8 g). The pH of the aqueous phase was adjusted to pH 2-3 using concentrated hydrochloric acid. The reaction mixture was then extracted with ethyl acetate (100 ml). The organic phase was separated off, dried over anhydrous Na₂SO₄ and concentrated in order to obtain a further 1.2 g of compound (j). The yield was 79%. Analytical data of (j): ¹H NMR (CDCl₃, 300 MHz) δ 0.84-1.94 (16H, m), 4.65 (3H, m, CH₂, CH), 5.24 (2H, s, CH₂), 6.88 (3H, m), 7.16 (1H, t, J=8.4 Hz), 7.34 (2H, d, J=8.1 Hz), 7.60 (1H, d, J=8.4 Hz), 7.73 (1H, d, J=8.4 Hz), ¹³C NMR (CDCl₃, 75 MHz): δ 14.6, 19.5, 23.6, 25.1, 31.3, 46.7, 66.7, 77.5, 91.6, 114.4, 120.9, 121.1, 122.7, 124.1, 128.6, 131.6, 131.9, 136.3, 141.9, 152.5, 158.6, 164.0; MS (FAB): found 567.5 (M⁺+Na), 545.5 (M⁺+1); IR (film, cm⁻¹) λₘₐₓ=3413, 2938, 2860, 1754, 1722, 1618, 1551, 1485, 1458, 1428, 1280, 1243, 1077, 1038, 1008, 989, 911, 871, 802, 747, 608.

### 6. Preparation of 1-{[(cyclohexyloxy)carbonyl]oxy}-1-iodo-ethane (k)

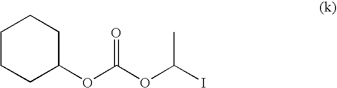

a) In a 100 ml round-bottomed flask, triphosgene (10 mmol, 39.0 g) was added at −40° C. to a suspension of acetaldehyde (360 mmol, 20 ml) and PhCH₂N⁺Et₃Cl⁻ (18 mmol, 4.1 g). The reaction mixture was stirred for 5 h. Excess triphosgene was removed under reduced pressure. The residue was distilled under reduced pressure and the distillate was collected at 41-42° C./4.2 mm Hg, which afforded 1-chlorocarbonyloxy-1-chloroethane (compound (m)) (21.2 g, 41.2% yield). Analytical data of (m): ¹H NMR (CDCl₃, 300 MHz) δ 1.85 (3H, d, J=5.7 Hz, CH₃), 6.42 (1H, q, J=5.7 Hz, CH).

b) In a 250 ml round-bottomed flask, compound (m) from step a) (10 ml) was added dropwise to a solution of cyclohexanol (91.5 mmol, 9.15 g) and pyridine (91.8 mmol, 7.38 ml) in CH₂Cl₂ (150 ml) cooled in an ice bath. The reaction mixture was stirred at room temperature for 16 h, washed with a saturated, aqueous NaCl solution, dried over anhydrous Na₂SO₄ and the solvent was subsequently distilled off. The residue was distilled under reduced pressure and the distillate was collected at 130-132° C./5 mm Hg, which afforded 1-{[(cyclohexyloxy)carbonyl]-oxy}-1-chloroethane (compound (n)) (16.7 g). Analytical data of (n): ¹H NMR (CDCl₃, 300 MHz) δ 1.20-1.53 (6H, m, CH₂), 1.70 (2H, m, CH₂), 1.78 (3H, d, J=6 Hz, CH₃), 1.89 (2H, m, CH₂), 4.64 (1H, m, CH), 6.39 (1H, q, J=6 Hz, CH).

c) Compound (n) from step b) (6.7 mmol, 1.4 g) was dissolved in 50 ml of MeCN in a 100 ml round-bottomed flask. NaI (26.8 mmol, 4.4 g) was added and the reaction mixture was stirred at 60° C. for 90 min. After it had been concentrated under reduced pressure, the residue was extracted with ether. The organic phase was separated off, dried over anhydrous Na₂SO₄ and purified by means of column chromatography in order to obtain the target compound (k) (810 mg, 40% yield). Analytical data of (k): ¹H NMR (CDCl₃, 300 MHz) δ 1.23-1.93 (10H, m, CH₂), 2.23 (3H, d, J=6 Hz, CH₃), 4.68 (1H, m, CH), 6.75 (1H, q, J=6 Hz, CH).

### 7. Preparation of Compound (o)

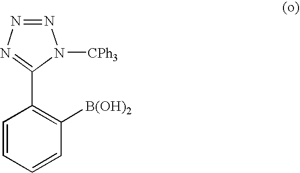

a) Benzonitrile (10.3 g, 100 mmol), NH₄Cl (6.9 g, 1.3 eq), NaN₃ (8.5 g, 1.3 eq) and LiCl (300 mg) were dissolved in 100 ml of DMF and the reaction mixture was stirred at 100° C. for 12 h. Subsequently, the major part of the solvent was removed under reduced pressure. The residue was rendered alkaline using 10% strength aqueous NaOH up to a pH of pH 12. After extraction with ethyl acetate, the aqueous phase was separated off and acidified to pH 2 using concentrated hydrochloric acid. The precipitate was filtered off using a Buchner funnel, washed with water and dried, which afforded compound (p)

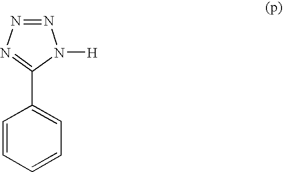

(13.5 g, melting point 208-209° C.). The yield was 96%. Analytical data of (p): ¹H NMR (d-DSMO, 300 MHz) δ 7.55-7.57 (3H, m), 8.01-8.03 (2H, m); ¹³C NMR (d-DMSO, 75 MHz): δ 129.5, 132.4, 134.8, 136.7, 160.7; MS (EI) m/z (%): 146 (M⁺, 42), 118 (100), 103 (17), 91 (46), 77 (32), 63 (48); IR (film, cm⁻¹) λₘₐₓ=3055, 2982, 2837, 2607, 2545, 1607, 1562, 1485, 1463, 1409, 1163, 1056, 1013, 725, 703, 686.

b) Compound (p) from step a) (6.6 g, 45 mmol) was dissolved in 20 ml of CH₂Cl₂ and treated with NEt₃ (8 ml, 1.3 eq). The reaction mixture was cooled to 0° C. in an ice bath and Ph₃CCl (13.2 g, 1.05 eq) was added in 3 portions in the course of 10 min. Subsequently, it was warmed to room temperature and stirred for 3 h. The reaction mixture was filtered, washed with water and dried in order to obtain compound (q)

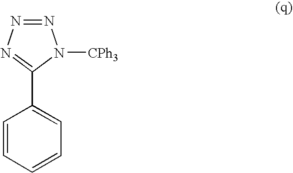

(16.5 g, melting point 163-164° C.). The yield was 94%. Analytical data of (q): ¹H NMR (CDCl₃, 300 MHz) δ 7.21-7.24 (6H, m), 7.37-7.39 (9H, m), 7.47-7.49 (3H, m), 8.19-8.20 (2H, m); ¹³C NMR (CDCl₃, 75 MHz): δ 83.0, 127.0, 127.5, 127.7, 128.3, 128.7, 130.3, 141.3, 164.0; IR (film, cm⁻¹) λₘₐₓ=3058, 1490, 1465, 1445, 1186, 1028, 874, 763, 748, 697, 635.

c) A solution of compound (q) from step b) (10 g, 25.8 mmol) in THF (30 ml) was temperature-controlled at −20° C. under argon (protective gas atmosphere). Subsequently, BuLi (1 M, 27 ml, 1.05 eq) was added. The temperature was increased to −5° C. and the mixture was stirred for 1 h. In the meantime, a large amount of a solid precipitated. The mixture was again cooled to −25° C. and B(OMe)₃ (4.3 ml, 1.5 eq) was added slowly by means of a syringe. Subsequently, the reaction mixture was allowed to warm to 20° C. and was stirred for half an hour. The solvent was reduced to ⅓ of the original amount under reduced pressure, a white solid forming. The solid was filtered off, washed with 20% THF in H₂O (40 ml) and water (40 ml) and dried, which afforded the target compound (o) (10.4 g). The yield was 94%. The compound (o) can be reused without further purification.

### 8. Preparation of Candesartan Cilexetil (C—C Coupling)

Examples 8-a1) to 8-a4) show 4 possible reaction conditions by means of which C—C coupling of the compound (j) from Example 5 can take place with the compound (o) from Example 7.

Example 8-b) describes the removal of the protective group with subsequent work-up.

a1) Compound (j) from Example 5 (2.5 g, 4.6 mmol), compound (o) from Example 7 (3.4 g, 1.2 eq) and Na₂CO₃ (1.46 g, 3 eq) were dissolved in 20 ml of toluene/water (7:3) and the system was flushed three times with argon. Subsequently, Pd(PPh₃)₄ (266 mg, 0.05 eq) was added and the reaction mixture was heated at 80° C. for 13 h. The reaction mixture was then extracted with ethyl acetate and purified by means of column chromatography (PE:ether, 3:2) in order

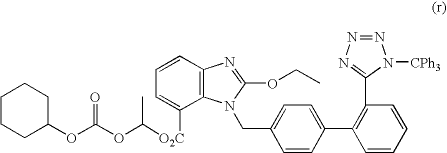

to obtain compound (r) (3.2 g, 82% yield). Analytical data of (r): ¹H NMR (CDCl₃, 400 MHz) δ 1.19-1.51 (11H, m), 1.67-1.71 (3H, m), 1.91 (2H, m), 4.59-4.65 (3H, m, CH₂ and CH), 5.56 (2H, q, J=16 Hz, CH₂), 6.78-7.47 (24H, m), 7.56 (1H, d, J=8 Hz), 7.76 (1H, d, J=8.4 Hz), 7.87 (1H, d, J=6.8 Hz); ¹³C NMR (CDCl₃, 100 MHz): δ 14.6, 19.5, 23.6, 25.1, 31.4, 47.0, 66.7, 77.5, 82.2, 91.7, 114.8, 120.8, 122.5, 124.0, 126.2, 126.3, 127.4, 127.6, 128.2, 129.4, 129.8, 130.2, 130.3, 130.6, 135.7, 140.0, 141.2, 141.8, 142.0, 152.5, 158.7, 163.9, 164.0; MS (FAB): found 875 (M⁺+Na), 853 (M⁺+1); IR (film, cm⁻¹) λₘₐₓ=2939, 2860, 1753, 1723, 1550, 1447, 1429, 1279, 1242, 1078, 1036, 909, 733, 699.

a2) NiCl₂(PPh₃)₂ (33 mg, 0.05 mmol), PPh₃ (26 mg, 0.1 mmol) were dissolved in 3 ml of DME (dimethoxyethane) or benzene under an argon protective gas atmosphere. Subsequently, butyllithium (0.13 ml, 0.2 mmol, 1.6 M in hexane) was added dropwise and the mixture was stirred for 10 min. Compound (j) from Example 5 (0.5 mmol), K₃PO₄ (1.5 mmol), compound (o) from Example 7 (1.1 mmol) were added and the reaction mixture was heated at 80° C. for 12 h. The reaction mixture was extracted twice with ethyl acetate and the organic phases were washed with water and saturated aqueous NaCl solution. The organic phase was separated off, dried over anhydrous Na₂SO₄ and purified by means of column chromatography.

a3) The reaction was carried out as described in Example 8-a2), instead of butyllithium DIBAH (diisobutyl-aluminum hydride) (0.045 ml, 0.2 mmol) alternatively being used.

a4) NiCl₂(PPh₃)₂ (33 mg, 0.05 mmol), PPh₃ (26 mg, 0.1 mmol) and zinc powder (55 mg, 0.85 mmol) were dissolved in 1 ml of THF under an argon protective gas atmosphere and the mixture was warmed to 50° C. for 1 h. Subsequently, compound (j) from Example 5 (0.5 mmol), K₃PO₄ (1.5 mmol) and compound (o) from Example 7 (1.1 mmol), and also 2 ml of THF were added. The reaction mixture was heated to reflux for 48 h and worked up as described under a2).

b) Compound (r) from step a) (3 g, 3.5 mmol) was dissolved in 51 ml of CH₂Cl₂:MeOH:1 N HCl (10:36:5.5) and the reaction mixture was stirred at room temperature for 3.5 h. Subsequently, the pH was adjusted approximately to pH 3 using saturated, aqueous NaHCO₃ and the major part of the solvent was removed under reduced pressure. The residue was extracted with ethyl acetate and purified by means of column chromatography (PE:AcOEt, 1:1) in order

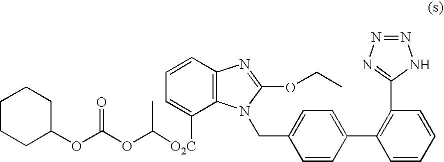

to obtain candesartan cilexetil (s) (2.05 g, melting point 128-129° C.). The yield was 95%. Analytical data of (s): ¹H NMR (CDCl₃, 300 MHz) δ 0.97-1.39 (11H, m), 1.46-1.48 (1H, m), 1.63 (2H, s, br), 1.78-1.82 (m, 2H), 3.92-4.00 (m, 1H), 4.28-4.36 (m, 1H), 4.46-4.52 (m, 1H), 5.55 (2H, dd, J=18, 20 Hz, CH₂), 6.55-6.60 (4H, m), 6.69-6.75 (2H, m), 6.81 (1H, t, J=7.8 Hz), 7.24 (1H, d, J=7.8 Hz), 7.39 (1H, d, J=7.5 Hz), 7.52-7.61 (2H, m), 7.93 (1H, d, J=8.1 Hz); ¹³C NMR (CDCl₃, 75 MHz): δ 14.4, 19.0, 23.4, 24.9, 31.2, 46.7, 67.7, 77.6, 91.7, 115.3, 120.6, 121.2, 123.3, 124.1, 124.9, 128.2, 129.4, 130.2, 130.4, 131.1, 136.1, 138.0, 139.5, 140.8, 152.2, 154.7, 157.7, 163.1; MS (FAB): found 633 (M⁺+Na) 611 (M⁺+1); IR (film, cm⁻¹) λₘₐₓ=3060, 2939, 2860, 1753, 1725, 1613, 1550, 1473, 1434, 1281, 1245, 1078, 1038, 992, 911, 730.

### 9. Preparation of a Novel Polymorphic Form of Candesartan Cilexetil

3 g of candesartan cilexetil are dissolved in 3 ml of dichloromethane. Subsequently, 50 ml of dimethyl ether are slowly added under reflux. In the case where a solid deposits, dichloromethane is added again until the solid redissolves. The clear solution is concentrated to approximately 5 ml under normal pressure and subsequently gradually cooled to room temperature, candesartan cilexetil of the novel polymorphic form being formed as a white solid.

The novel polymorphic form can be prepared by the process just described.

The novel polymorphic form can be described by one or more of the following physical parameters:
signals in the X-ray powder diffractogram expressed in 2θ at 7.32; 8.20; 9.10; 14.68; 18.88; 24.18°, where the respective relative signal intensities can be 100; 29.6; 20.2; 45.2; 20.5; 11.4 (cf. FIGS. 1a, 2);
spacings of the lattice planes d determined by means of XRD=12.065; 10.773; 9.711; 6.029; 4.696; 3.678 Å (Angstroms); (cf. FIG. 2)
a melting point determined by means of DSC at approximately 130.7° C. (cf. FIG. 3, curve d); and
a characteristic absorption band in the IR spectrum at ν=1733 cm⁻¹ (cf. FIG. 4).

With the aid of the examples described, it has been shown how candesartan cilexetil is obtainable in the manner according to the invention starting from any desired one of the intermediates according to the invention.

Furthermore, a novel form of candesartan cilexetil and a process for its preparation have been described for the first time. The examples serve only for the illustration of the invention, without restricting it according to scope.

1. Process for the preparation of candesartan, of a candesartan salt, or of a candesartan ester or of a protected form of candesartan, of a candesartan salt, or of a candesartan ester, in particular candesartan cilexetil, which comprises the following steps:
(a) preparation and reaction of a compound of the formula (I) 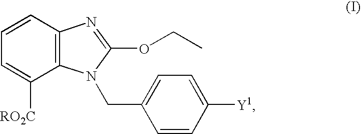
in which
R is hydrogen, an unsubstituted or substituted alkyl or aryl radical, preferably methyl or (cyclo-hexyloxycarbonyloxy)ethyl,
Y¹ is a group which is able to enter into a coupling reaction, into which a group Y² further enters, with formation of a C—C bond,
with a compound of the formula (II) containing the group Y² 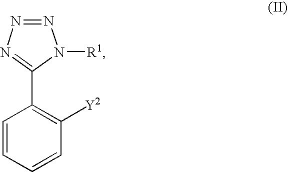
in which R¹ is a tetrazolyl protective group or hydrogen, with formation of candesartan, of a protected form of candesartan or of a candesartan ester or candesartan cilexetil, and optionally
(b) conversion to candesartan, candesartan cilexetil or to a salt.

2. Process according to claim 1, where R=methyl.

3. Process according to claim 1, where R is an alkyl radical, preferably methyl, and where step (b) comprises a transesterification of the ester resulting from step (a).

4. Process according to claim 3, where step (b) comprises the hydrolysis of the ester resulting from step (a) by means of treatment with NaOH in EtOH.

5. Process according to claim 4, where step (b) furthermore comprises the reaction of candesartan in the form of the free carboxylic acid with a compound of the formula (IV) 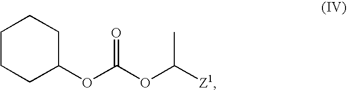
in which Z¹ is a leaving group, preferably a halogen, preferably iodine,
with formation of candesartan cilexetil,
preferably in the presence of NaI and K₂CO₃.

6. Process according to claim 1, where R¹ is selected from one of the following groups: hydrogen, tert-butyl and triphenylmethyl, preferably triphenyl-methyl.

7. Process according to claim 1, where Y¹ is selected from one of the following functional groups:
B(OR⁴)₂, where each of the radicals R⁴ independently of one another represents
hydrogen, alkyl, aryl or alkylaryl, preferably hydrogen,
a trialkyltin radical, or
a magnesium(II) halide radical or
halogen, preferably bromine,
and where, if Y² represents a halogen, Y¹ represents B(OR⁴), a trialkyltin radical or a magnesium(II) halide radical and conversely.

8. Process according to claim 1, where Y² is selected from one of the following groups:
halogen, preferably bromine,
a trialkyltin radical,
a magnesium(II) halide radical or
B(OR⁴)₂, where each of the radicals R⁴ independently of one another represents
hydrogen, alkyl, aryl or alkylaryl, preferably hydrogen,
and where, if Y¹ represents a halogen, Y² represents B(OR⁴), a trialkyltin or a magnesium(II) halide radical and conversely.

9. Process according to claim 1, where one or more catalysts, preferably comprising one or more transition metals, are employed.

10. Process according to claim 9, where the catalyst or the catalysts is or are selected from MnCl₂, CrCl₃, FeCl₂, Fe(acac)₃, FeCl₃, Fe(salen)Cl, CoCl₂(dppe), CoCl₂(dpph), Co(acac)₂, CoCl₂(dppb), Pd(PPh₃)₄ or NiCl₂(PPh₃)₂.

11. Process according to claim 9, where the catalyst is employed together with an activator and/or stabilizer.

12. Process according to claim 10, where the catalyst or the catalysts is/are selected from the group consisting of the phosphane-free, preferably iron-containing catalysts.

13. Process according to claim 9, where one or more of the following solvents is or are used: THF, THF/NMP, Et₂O, DME, benzene, toluene.

14. Process for the preparation of a compound having the formula (I) defined in claim 1, which comprises the following steps:
preparation and reaction of a compound of the formula (III) 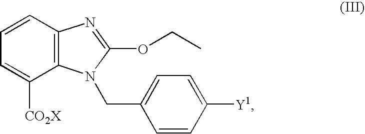
in which
Y¹ is a group which is able to enter into a coupling reaction, into which a group Y² further enters, with formation of a C—C bond or Y¹B(OR⁴)₂, where each of the radicals R⁴ independently of one another represents hydrogen, alkyl, aryl or alkylaryl, preferably hydrogen,
a trialkyltin radical, or
a magnesium(II) halide radical or
halogen, preferably bromine,
and where, if Y² represents a halogen, Y¹ represents B(OR⁴), a trialkyltin radical or a magnesium(II) halide radical and conversely,
X is a group which is able to enter into a reaction, into which a group Z¹ further enters, with formation of an O—C bond,
with a compound of the formula (IV) 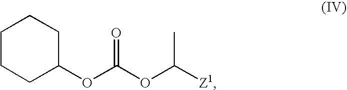
in which
Z¹ is a leaving group,
with formation of a compound of the formula (I).

15. Process according to claim 14, where X is an alkali metal or preferably hydrogen and/or Z¹ represents a halogen, preferably iodine.

16. Process for the preparation of a compound having the formula (III) defined in claim 14, which comprises the following step:
preparation and deprotection of a compound of the formula (V) 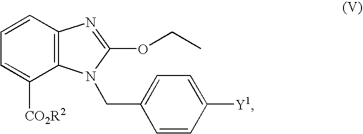
in which
Y¹ is a group which is able to enter into a coupling reaction, into which a group Y² further enters, with formation of a C—C bond or Y¹B(OR⁴)₂, where each of the radicals R⁴ independently of one another represents hydrogen, alkyl, aryl or alkylaryl, preferably hydrogen,
a trialkyltin radical, or
a magnesium(II) halide radical or
halogen, preferably bromine,
and where, if Y² represents a halogen, Y¹ represents B(OR⁴), a trialkyltin radical or a magnesium(II) halide radical and conversely,
R² is a group replaceable by X with formation of a compound of the formula (III), where X is a group which is able to enter into a reaction, into which a group Z¹ further enters, with formation of an O—C bond or is an alkali metal or preferably hydrogen and/or Z¹ represents a halogen, preferably iodine.

17. Process according to claim 16, where R² is selected from one of the following functional groups: substituted or unsubstituted C₁-C₆-lower alkyl, benzyl, or aryl, preferably ethyl (CH₂CH₃) and even more preferably methyl (CH₃).

18. Process for the preparation of a compound having the formula (V) defined in claim 16, which comprises the following step:
preparation and reaction of a compound of the formula (VI) 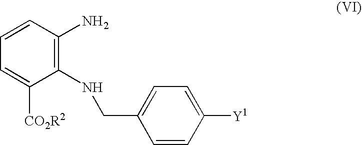
in which
Y¹ is a group which is able to enter into a coupling reaction, into which a group Y² further enters, with formation of a C—C bond or Y¹B(OR⁴)₂, where each of the radicals R⁴ independently of one another represents hydrogen, alkyl, aryl or alkylaryl, preferably hydrogen,
a trialkyltin radical, or
a magnesium(II) halide radical or
halogen, preferably bromine,
and where, if Y² represents a halogen, Y¹ represents B(OR⁴), a trialkyltin radical or a magnesium(II) halide radical and conversely, and
R² is a group replaceable by X with formation of a compound of the formula (III), where X is a group which is able to enter into a reaction, into which a group Z¹ further enters, with formation of an O—C bond or is an alkali metal or preferably hydrogen and/or Z¹ represents a halogen, preferably iodine selected from one of the following functional groups: substituted or unsubstituted C₁-C₆-lower alkyl, benzyl, or aryl, preferably ethyl (CH₂CH₃) and even more preferably methyl (CH₃),
with a carbonylating reagent or preferably C(OEt)₄, with formation of a compound of the formula (V).

19. Process according to claim 18, where Ac₂O is further used in the reaction.

20. Process for the preparation of a compound of the formula (VI) as defined in claim 18, which comprises the following step:
preparation of a compound of the formula (VII) 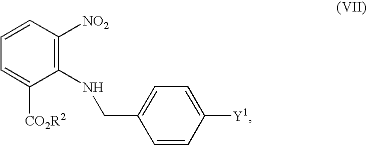
in which
Y¹ is a group which is able to enter into a coupling reaction, into which a group Y² further enters, with formation of a C—C bond or Y¹B(OR⁴)₂, where each of the radicals R⁴ independently of one another represents hydrogen, alkyl, aryl or alkylaryl, preferably hydrogen,
a trialkyltin radical, or
a magnesium(II) halide radical or
halogen, preferably bromine,
and where, if Y² represents a halogen, Y¹ represents B(OR⁴), a trialkyltin radical or a magnesium(II) halide radical and conversely, and
R² is a group replaceable by X with formation of a compound of the formula (III), where X is a group which is able to enter into a reaction, into which a group Z¹ further enters, with formation of an O—C bond or is an alkali metal or preferably hydrogen and/or Z¹ represents a halogen, preferably iodine selected from one of the following functional groups: substituted or unsubstituted C₁-C₆-lower alkyl, benzyl, or aryl, preferably ethyl (CH₂CH₃) and even more preferably methyl (CH₃),
and conversion of the nitro group present therein to an amine group.

21. Process according to claim 20, where the conversion of the nitro group to the amine group can be brought about with the aid of base metals, catalytic hydrogenation, by electrolytic routes or preferably with the aid of SnCl₂.

22. Process for the preparation of a compound of the formula (VII) as defined in claim 20, which contains the following step:
preparation and deprotection of a compound of the formula (VIII) 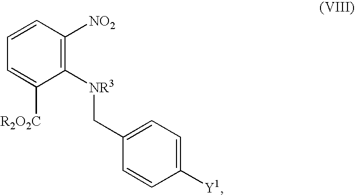
in which
Y¹ is a group which is able to enter into a coupling reaction, into which a group Y² further enters, with formation of a C—C bond or Y¹B(OR⁴)₂, where each of the radicals R⁴ independently of one another represents hydrogen, alkyl, aryl or alkylaryl, preferably hydrogen,
a trialkyltin radical, or
a magnesium(II) halide radical or
halogen, preferably bromine,
and where, if Y² represents a halogen, Y¹ represents B(OR⁴), a trialkyltin radical or a magnesium(II) halide radical and conversely, and
R² is a group replaceable by X with formation of a compound of the formula (III), where X is a group which is able to enter into a reaction, into which a group Z¹ further enters, with formation of an O—C bond or is an alkali metal or preferably hydrogen and/or Z¹ represents a halogen, preferably iodine selected from one of the following functional groups: substituted or unsubstituted C₁-C₆-lower alkyl, benzyl, or aryl, preferably ethyl (CH₂CH₃) and even more preferably methyl (CH₃ and
R³ is a protective group replaceable by H,
with formation of a compound of the formula (VII)

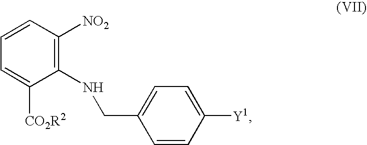

23. Process according to claim 22, where R³ is a carboxy-alkyl group, preferably a carboxy-tert-butyl group (—COOC(CH₃)₃).

24. Process for the preparation of a compound of the formula (VIII) as defined in claim 22, which has the following step:
preparation and reaction of a compound of the formula (IX) 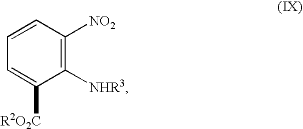
in which
R² is a group replaceable by X with formation of a compound of the formula (III), where X is a group which is able to enter into a reaction, into which a group Z¹ further enters, with formation of an O—C bond or is an alkali metal or preferably hydrogen and/or Z¹ represents a halogen, preferably iodine selected from one of the following functional groups: substituted or unsubstituted C₁-C₆-lower alkyl, benzyl, or aryl, preferably ethyl (CH₂CH₃) and even more preferably methyl (CH₃, and
R³ is a protective group replaceable by H or a carboxy-alkyl group, preferably a carboxy-tert-butyl group (—COOC(CH₃)₃)
with a compound of the formula (X) 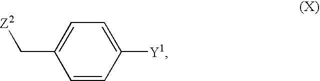
in which
Y¹ is a group which is able to enter into a coupling reaction, into which a group Y² further enters, with formation of a C—C bond or Y¹B(OR⁴)₂, where each of the radicals R⁴ independently of one another represents hydrogen, alkyl, aryl or alkylaryl, preferably hydrogen,
a trialkyltin radical, or
a magnesium(II) halide radical or
halogen, preferably bromine,
and where, if Y² represents a halogen, Y¹ represents B(OR⁴), a trialkyltin radical or a magnesium(II) halide radical and conversely, and
Z² is a leaving group,
with formation of a compound of the formula (VIII).

25. Process according to claim 24, where Z² is selected from one of the following functional groups: Cl, I and preferably Br.

26. (canceled)

27. (canceled)

28. (canceled)

29. (canceled)

30. (canceled)

31. (canceled)

32. Compound of the formula (I) as defined in claim 1.

33. Compound according to claim 32, where Y¹ is ═Br.

34. Compound of the formula (III) as defined in claim 14.

35. Compound according to claim 34, where Y¹ is ═Br.

36. Compound of the formula (V) as defined in claim 16.

37. Compound according to claim 36, where Y¹=Br and R² is a methyl or C₃-C₆-lower alkyl group.

38. Compound of the formula (VI) as defined in claim 18.

39. Compound according to claim 38, where Y¹=Br and R² is a methyl or C₃-C₆-lower alkyl group.

40. Compound of the formula (VIII) as defined in claim 22.

41. Compound according to claim 40, where
Y¹=Br,
R² is a methyl or C₃-C₆-lower alkyl group, and
R³ is a carboxyalkyl group, preferably a carboxy-tert-butyl group (—COOC(CH₃)₃).

42. Use of a compound according claim 32 for the preparation of candesartan or of a candesartan ester, in particular of candesartan cilexetil.

43. (canceled)

44. (canceled)

45. (canceled)

46. (canceled)

47. (canceled)

48. (canceled)

49. (canceled)

50. (canceled)
